(19) United States
(12) Patent Application Publication
(10) Pub. No.: US 2019/0194618 A1
(43) Pub. Date: Jun. 27, 2019

(54) METHOD FOR MEASURING IMMUNOGENICITY OF PROTEIN AGENT

(71) Applicant: MOGAM INSTITUTE FOR BIOMEDICAL RESEARCH, Yongin-si, Gyeonggi-do (KR)

(72) Inventors: Ok Jae LIM, Yongin-si (KR); Duck Hyang SHIN, Yongin-si (KR)

(73) Assignee: MOGAM INSTITUTE FOR BIOMEDICAL RESEARCH, Yongin-si, Gyeonggi-do (KR)

(21) Appl. No.: 16/329,403

(22) PCT Filed: Aug. 31, 2017
(86) PCT No.: PCT/KR2017/009522

§ 371 Date: Feb. 28, 2019

(30) Foreign Application Priority Data
Sep. 1, 2016 (KR) 10-2016-0112817

(51) Int. Cl. C12N 5/0783; C12N 5/0784; C12N 5/00

(52) U.S. Cl. CPC C12N 5/0636; C12N 5/0639; C12N 5/0018; C12N 2501/22; C40B 40/02; C12N 2501/25; C12N 2501/2301; C12N 2501/2306; C12N 2501/2304

(57) ABSTRACT

A method for determining immunogenicity of a protein agent. The method includes constructing a library of peripheral blood mononuclear cells having various HLA-DRB1 genotypes; culturing peripheral blood mononuclear cell CD14+ monocyte-derived immature dendritic cells for each genotype in a medium containing a protein to be measured, GM-CSF, IL-4, TNF-α, IL-1β, IL-6 and PGF₂ to prepare mature dendritic cells; removing CD8+ T cells from the peripheral blood mononuclear cells for each genotype to prepare CD8+ T cell-free peripheral blood mononuclear cells; co-culturing the mature dendritic cells and the CD8+ T cell-free peripheral blood mononuclear cells at a cell count ratio of approximately 1:5 to 1:20; and quantifying the CD4+ T cells proliferated by co-cultivation per genotype.

### TECHNICAL FIELD

The present invention relates to a method for determining immunogenicity of a protein agent.

### BACKGROUND ART

For purpose of treating a variety of diseases, diverse protein formulations are under research and development and used, in particular, there are a number of clinical cases with very successful effects for therapeutic antibodies. Representative examples of such therapeutic antibodies may include adalimumab (Humira®), etanercept (Enbrel®), etc. targeting inflammatory diseases such as rheumatoid arthritis, rituximab (Mabthera®) targeting lymphoma, trastuzumab (Herceptin®) targeting the breast cancer, or the like.

A number of cases of causing immune response not intended in clinical examples with application of antibody therapeutics have been reported. In a case of first generation antibody therapeutics, an immune response to an antibody derived from mice, in particular, an immune response against a therapeutic antibody administered, that is, an anti-drug antibody (ADA) reaction has occurred due to a difference in DNA sequences relative to human antibody. In order to overcome this problem, a chimeric antibody having a constant region substituted by the human antibody exhibited quite improved immune response. Further, possibility of immunogenicity induction due to the difference in DNA sequences was greatly reduced with the development of a technique of humanizing even a variable region. However, even for adalimumab (Humira®) developed as a complete human antibody in practice, it was found that immune response occurs in up to 26% of patients administered with the above medication as a result of clinical application.

In general, effects of a drug are proportional to a blood concentration of the same. If an immune response to a drug administered for therapeutic purposes is induced, the efficacy of the drug is lowered as the half-life of the drug is decreased. Further, the induced immune response to the drug may cause side effects such as fever and inflammatory reaction.

For an assessment of immunogenicity in regard to protein drugs (including antibody medicaments), a method of collecting peripheral blood from the patient to check whether ADA was created or not, has been used until recently. However, this test method cannot assess immunogenicity unless the protein drug of interest is under clinical application, thereby having a disadvantage in that the immunogenicity cannot be predicted in a development phase of the drug. In consideration of huge costs and time involved in the development of a drug till the entry on the clinical phase, the fact that the immunogenicity is not determined till the entry on the clinical phase is the major obstacle in development of protein drugs.

Therefore, it is urgently required to develop an improved method for determination of immunogenicity which can predict an extent of immunogenicity possibly induced by a protein formulation in a drug development phase so as to select the protein with low immunogenicity in advance. Such a method for determination of immunogenicity may contribute to pre-selection of a desired protein that has high medical effects and duration with reduced side effects, thereby greatly increasing development efficiency of the protein formulation.

### PRIOR ART DOCUMENT

### Patent Document

Korean Patent Registration Publication No. 10-1047207 (Published on Jun. 30, 2011)

### DISCLOSURE

### Technical Problem

Accordingly, an object of the present invention is to provide a method for determination of immunogenicity which includes predicting an extent of immunogenicity induced by a protein formulation (that is, ‘protein agent’) in a drug development phase, thereby selecting the desired protein with low immunogenicity in advance.

### Technical Solution

(1) A method for determining immunogenicity of a protein agent, including: constructing a library of peripheral blood mononuclear cells having various HLA-DRB1 genotypes; culturing peripheral blood mononuclear cell CD14+ monocyte-derived immature dendritic cells for each genotype in a medium containing the protein to be measured, GM-CSF, IL-4, TNF-α, IL-1β, IL-6 and PGF₂ to prepare mature dendritic cells; removing CD8+ T cells from the peripheral blood mononuclear cells for each genotype to prepare CD8+ T cell-free peripheral blood mononuclear cells; co-culturing the mature dendritic cells and the CD8+ T cell-free peripheral blood mononuclear cells at a cell count ratio of 1:5 to 1:20; and quantifying the number of CD4+ T cells proliferated by the co-cultivation per genotype.

(2) The method according to the above (1), wherein the peripheral blood mononuclear cell library includes 70% or more of human HLA-DRB1 genetic polymorphism.

(3) The method according to the above (1), wherein the peripheral blood mononuclear cells are extracted from the blood collected by Leukoreduction system chambers (LRSCs).

(4) The method according to the above (1), wherein the immature dendritic cells are prepared by culturing the CD14 + mononuclear cells in a medium containing GM-CSF and IL-4.

(5) The method according to the above (1), wherein the CD8+ T cell-free peripheral blood mononuclear cells provided for the co-cultivation are stained with a fluorescent dye, and the quantification includes quantifying the number of CD4+ T cells having a fluorescent signal intensity decreased after the co-cultivation.

(6) The method according to the above (1), wherein the medium in the co-cultivation is a serum-free medium further containing L-glutamine, human serum albumin, streptomycin sulfate and gentamicin sulfate.

(7) The method according to the above (6), wherein the streptomycin sulfate is contained in an amount of 50 μg/ml, and the gentamicin sulfate is contained in an amount of 10 μg/ml.

(8) The method according to the above (1), wherein the medium in the step of preparing the mature dendritic cells further includes Ca(NO₃)₂.4H₂O, KCl, MgSO₄ (anhydrous), NaCl, Na₂HPO₄ (anhydrous), D-glucose, Glutathione (reduced), Phenol red, L-arginine, L-asparagine (free base), L-aspartic acid, L-cystine⋅2HCl, L-glutamic acid, L-glutamine, glycine, L-histidine (free base), L-hydroxyproline, L-isoleucine, L-leucine, L-lysine⋅HCl, L-methionine, L-phenylalanine, L-proline, L-serine, L-threonine, L-tryptophan, L-tyrosine⋅2Na2H₂O, L-valine, biotin, D-Ca⋅pantothenate, choline chloride, folic acid, i-inositol, para-aminobenzoic acid, niacinamide, pyridoxine⋅HCl, riboflavin, thiamine⋅HCl and vitamine B12, and further includes 10% (vol/vol) FBS.

(9) The method according to the above (4), wherein the medium used in the step of preparing the immature dendritic cells further include Ca(NO₃)₂.4H₂O, KCl, MgSO₄ (anhydrous), NaCl, Na₂HPO₄ (anhydrous), D-glucose, Glutathione (reduced), Phenol red, L-arginine, L-asparagine (free base), L-aspartic acid, L-cystine⋅2HCl, L-glutamic acid, L-glutamine, glycine, L-histidine (free base), L-hydroxyproline, L-isoleucine, L-leucine, L-lysine⋅HCl, L-methionine, L-phenylalanine, L-proline, L-serine, L-threonine, L-tryptophan, L-tyro sine⋅2Na2H₂O, L-valine, biotin, D-Ca⋅pantothenate, choline chloride, folic acid, i-inositol, para-aminobenzoic acid, niacinamide, pyridoxine⋅HCl, riboflavin, thiamine⋅HCl and vitamine B12, and further includes 10% (vol/vol) FBS.

(10) A kit for measuring immunogenicity of a protein agent, including a library of peripheral blood mononuclear cells including 70% or more of human HLA-DRB1 genetic polymorphisms.

### Advantageous Effects

According to the method for determination of immunogenicity of the present invention, it is possible to predict relative immunogenicity of protein agent candidates under development at high accuracy before the entry on a clinical phase.

According to the method for determination of immunogenicity of the present invention, it is possible to predict relative immunogenicity of protein agent candidates under development by in vitro (ex vivo) experiments.

According to the method for determination of immunogenicity of the present invention, it is possible to increase efficiency of protein agent development.

The method for determination of immunogenicity of the present invention may be conducted at a low cost.

### DESCRIPTION OF DRAWINGS

FIG. 1 illustrates HLA-DRB1 genotypes of peripheral blood mononuclear cell library of the present invention.

FIG. 2 illustrates a proliferation index (PI) drawn by the measuring method of the present invention.

FIG. 3 illustrates a response index (RI) drawn by the measuring method of the present invention.

FIG. 4 illustrates a relationship between the RI drawn by the measuring method of the present invention and an extent of ADA generation reported in the clinical phase.

### BEST MODE

The present invention discloses a method for determining immunogenicity of a protein agent, and more particularly, a method for determining immunogenicity of a protein agent including: constructing a library of peripheral blood mononuclear cells having various HLA-DRB1 genotypes; culturing peripheral blood mononuclear cell CD14+ monocyte-derived immature dendritic cells for each genotype in a medium containing a protein to be measured, GM-CSF, IL-4, TNF-α, IL-1β, IL-6 and PGF₂ to prepare mature dendritic cells; removing CD8+ T cells from the peripheral blood mononuclear cells for each genotype to prepare CD8+ T cell-free peripheral blood mononuclear cells; co-culturing the mature dendritic cells and the CD8+ T cell-free peripheral blood mononuclear cells at a cell count ratio of 1:5 to 1:20; and quantifying the number of CD4+ T cells proliferated by the co-cultivation per genotype, whereby relative immunogenicity of a protein agent candidate under development may be predicted with high accuracy before the entry on a clinical phase, thus increasing the development efficiency of protein agents.

Hereinafter, the present invention will be described in detail.

The method for determining immunogenicity of protein agent according to the present invention includes the steps of:

constructing a library of peripheral blood mononuclear cells having various HLA-DRB1 genotypes;

culturing peripheral blood mononuclear cell CD14+ monocyte-derived immature dendritic cells for each genotype in a medium containing the protein to be measured, GM-CSF, IL-4, TNF-α, IL-1β, IL-6 and PGF₂ to prepare mature dendritic cells;

removing CD8+ T cells from the peripheral blood mononuclear cells for each genotype to prepare CD8+ T cell-free peripheral blood mononuclear cells;

co-culturing the mature dendritic cells and the CD8+ T cell-free peripheral blood mononuclear cells at a cell count ratio of 1:5 to 1:20; and

quantifying the number of CD4+ T cells proliferated by the co-cultivation per genotype.

The method for determining immunogenicity of a protein agent according to the present invention may include constructing a library of peripheral blood mononuclear cells having various HLA-DRB1 genotypes.

The peripheral blood mononuclear cells (PBMCs) are found in blood and each cell has a single round nucleus. The peripheral blood mononuclear cells may include lymphocytes such as T cells, B cells, NK cells, etc., and monocytes. The monocyte is a cell possibly differentiating into, for example, dendritic cells, macrophages.

HLA-DRB1 gene is a gene encoding HLA class II histocompatibility antigen protein, that is, DRB1. The HLA-DRB1 gene is mostly expressed on antigen presenting cells (APCs) and has an important role in activating T cells by presenting an extracellular protein on the surface of APC. The HLA-DRB1 gene is present in a great number of polymorphisms, and according to the genotype of HLA-DRB1, whether or not to generate of anti-drug antibody (ADA) against a specific protein agent and the extent of generation may be varied.

In order to accurately determine the immunogenicity of a specific protein agent, it is preferable to measure the immunogenicity using a library of peripheral blood mononuclear cells having various HLA-DRB1 genotypes. According to an embodiment, the immunogenicity of the protein agent of interest is measured for each genotype, and after repeatedly measuring the same immunogenicity with respect to various genotypes, all of the measured immunogenicity values are collected to deduce a measured value of immunogenicity of the protein of interest. Herein, in a case of predicting the immunogenicity in an actual clinical field from the measured values, an appearance frequency of the specific HLA-DRB1 genotypes may be considered. For example, weighted values may be applied to the measured values depending on the appearance frequency of specific HLA-DRB1 genotypes in specific races and/or countries to deduce customized immunogenicity measurement values with improved accuracy in terms of races and/or countries.

According to one embodiment of the present invention, the peripheral blood mononuclear cell library may include 50% or more of peripheral blood mononuclear cells with the human HLA-DRB1 genetic polymorphism. According to a more particular embodiment, the library may include 70% or more (e.g., 75% or more, 79% or more, or 80% or more) of peripheral blood mononuclear cells with the human HLA-DRB1 genetic polymorphism. According to one embodiment of the present invention, the peripheral blood mononuclear cell library may be formed by selecting specific PBMC of a donor, which can cover 50% or more (e.g., 70% or more, 75% or more, 79% or more, or 80% or more) of global population based on a frequency distribution of HLA-DRB1 allele.

The method for determining immunogenicity of a protein agent according to the present invention may include culturing CD14+ mononuclear cell-derived dendritic cells of peripheral blood mononuclear cells given for each genotype of a donor in a medium containing the protein to be measured, granulocyte-macrophage colony-stimulating factor (GM-CSF), interleukin-4 (IL-4), tumor necrosis factor alpha (TNF-α), interleukin-1β (IL-1β), interleukin-6 (IL-6) and prostaglandin E₂ (PGE₂), thus to prepare mature dendritic cells.

CD14+ mononuclear cells may be obtained by selectively extracting CD14 expressing mononuclear cells among the peripheral blood mononuclear cells. Such selective extraction of CD14 expressing mononuclear cells may include different methods, for example, using a magnetic-activated cell sorting (MACS) device.

The immature dendritic cells (immature DC) are cells present in an intermediate step for maturation of the peripheral blood mononuclear cells (PBMCs) into mature dendritic cells (mature DCs). The immature dendritic cells may uptake antigen, and the uptaken antigen is treated in the cells and then loaded on the HLA class II, thus to be presented on the surface of the mature dendritic cells. The mature dendritic cells to present the antigen on the surface thereof may stimulate T cells and induce proliferation of CD4+ T cells capable of stimulating B cells that may generate and secrete an antibody corresponding to the above antigen.

A target protein refers to a protein with immunogenicity to be determined, and may include diverse protein agents such as antibody formulations.

The method for determining immunogenicity of a protein agent according to the present invention may include removing CD8+ T cells from peripheral blood mononuclear cells of a donor who is selected for each genotype to prepare CD8+ T cell-free peripheral blood mononuclear cells.

The CD8+ T cell-free peripheral blood mononuclear cells may be obtained by selectively removing T cells that express CD8 in the peripheral blood mononuclear cells. There are various methods to selectively remove CD8+ T cell, for example, the magnetic-activated cell sorting (MACS) device may be used.

The method for determining immunogenicity of a protein agent according to the present invention may include co-culturing the mature dendritic cells and the CD8+ T cell-free peripheral blood mononuclear cells at a cell count ratio of 1:5 to 1:20 (e.g., 1:10) in a medium. According to one embodiment, the number of the mature dendritic cells may range from 2×10⁴ to 1×10⁵ cells/100 μl/well, and the number of the CD8+ T cell-free peripheral blood mononuclear cells may range from 2×10⁵ to 1×10⁶ cells/100 μl/well. For example, the number of the mature dendritic cells may be 5×10⁴ cells/100 μl/well, and the number of the CD8+ T cell-free peripheral blood mononuclear cells may be 5×10⁵ cells/100 μl/well.

Through the co-cultivation as described above, it is possible to induce proliferation of CD4+ T cells that recognize the antigen presented on the mature dendritic cells. In this case, if the CD8+ T cell-free peripheral blood mononuclear cells are not used as cells for co-cultivation with the mature dendritic cells (for example, when using whole peripheral blood mononuclear cells or only CD4+ T cells after extracting the same), immunogenicity determination accuracy may be reduced due to non-specific reaction or the like.

The method for determination of immunogenicity according to the present invention may include quantifying the number of CD4+ T cells proliferated by the co-cultivation. According to the present invention, the extent of proliferation of CD4+ T cells by the mature dendritic cells to present a target protein, that is, the number of proliferated CD4+ T cells has a high correlation to an extent of generation of an anti-drug antibody (ADA) to the target protein when administering the protein in vivo.

According to one embodiment of the present invention, the peripheral blood mononuclear cells may be extracted from blood. The blood may be obtained through different routes, and according to the preferred embodiment of the present invention, the blood may be collected from a leukocyte reduction filter, that is, a Leukoreduction system chambers (LRSCs). When the Leukoreduction system chamber (LRSC) is used as a source of blood, there is an advantage of easily supplying the blood.

According to one embodiment of the present invention, the immature dendritic cells may be prepared from CD14+ mononuclear cells isolated from the peripheral blood mononuclear cells. For example, the immature dendritic cells may be obtained by treating the CD14+ mononuclear cells isolated from the peripheral blood mononuclear cells with a granulocyte-macrophage colony stimulating factor (GM-CSF) as well as interleukin-4 (IL-4).

CD8+ T cell-free peripheral blood mononuclear cells used for co-cultivation with the mature dendritic cells may be stained with a fluorescent dye. For example, the fluorescent dye may be combined with intracellular components through covalent bonds to generate a fluorescent signal. If the cell stained with the fluorescent dye is proliferated, the fluorescent signals of each of such the proliferated cells are decreased by half, compared to before the proliferation. Therefore, according to one embodiment of the present invention, it is possible to quantify the number of proliferated CD4+ T cells by quantifying the number of CD4+ T cells with decreased fluorescent signals.

According to one embodiment of the present invention, the medium used in the co-cultivation may be a serum-free medium further containing, for example, L-glutamine, human serum albumin (HSA), streptomycin sulfate and gentamicin sulfate. According to a specific embodiment, the medium may include streptomycin sulfate at a concentration of 50 μg/ml and gentamicin sulfate at a concentration of 10 μg/ml. According to the present invention, when using the above medium in the co-cultivation step of T cells and dendritic cells, background noise is low while accelerating antigen-specific proliferation, thereby improving accuracy of immunogenicity determination.

According to one embodiment, the culture medium in both steps of preparing the immature dendritic cells and the immature dendritic cells may further include, for example, Ca(NO₃)₂.4H₂O, KCl, MgSO₄ (anhydrous), NaCl, Na₂HPO₄ (anhydrous), D-glucose, Glutathione (reduced), Phenol red, L-arginine, L-asparagine (free base), L-aspartic acid, L-cystine⋅2HCl, L-glutamic acid, L-glutamine, glycine, L-histidine (free base), L-hydroxyproline, L-isoleucine, L-leucine, L-lysine⋅HCl, L-methionine, L-phenylalanine, L-proline, L-serine, L-threonine, L-tryptophan, L-tyrosine⋅2Na2H₂O, L-valine, biotin, D-Ca⋅pantothenate, choline chloride, folic acid, i-inositol, para-aminobenzoic acid, niacinamide, pyridoxine⋅HCl, riboflavin, thiamine⋅HCl and vitamine B12, and may further include 10% (vol/vol) FBS. Using the medium as descried above in the steps of preparing the immature dendritic cells and the mature dendritic cells, it is possible to desirably improve differentiation efficiency of the dendritic cells, and enhance the accuracy of immunogenicity determination.

A kit for measuring immunogenicity of a protein agent according to the present invention may include a library of peripheral blood mononuclear cells containing 50% or more (e.g., 70% or more, 75% or more, 79% or more, or 80% or more) of human HLA-DRB1 polymorphism. According to one embodiment of the present invention, the kit for measurement of immunogenicity according to the present invention may include the library produced by selecting specific PBMC of a donor that can cover 50% or more (e.g., 70% or more, 75% or more, 79% or more, or 80% or more) of global population based on the frequency distribution of HLA-DRB1 allele.

Hereinafter, the present invention will be described in more detail by means of the following examples. These examples are provided for more concretely describing the present invention, however, the scope of subject matters to be protected is duly not limited to those illustrated in the examples.

### EXAMPLE 1-1

### Preparation of Human Peripheral Blood Mononuclear Cells (PBMCs)

1. PBMC Preparation

A Leukoreduction system chamber (LRSC), that is, a leukocyte removal filter was prepared after sterilizing an outside thereof with an alcohol swab. 20 mL of elution buffer (PBS supplemented with 0.6 (vol/vol) ACD) was poured into a 50 ml tube. After cutting off inlet and outlet line tubes connected to the LRSC with a pair of sterile scissors, the blood was collected in the tube containing the elution buffer. 30 mL of elution buffer was further added thereto, followed by washing and collecting the blood.

After placing 15 mL Ficoll-Paque™ in a new 50 mL tube, the blood diluted with buffer was overlaid slowly by 30 mL. After centrifugation at 18000 rpm for 20 minutes, a layer containing PBMC was collected and a washing buffer (PBS supplemented with 0.6% (vol/vol) ACD and 2% (vol/vol) FEB) was added thereto, followed by further centrifugation at 1500 rpm for 10 minutes. After discarding a supernatant, the remaining product was suspended in 50 mL of the washing buffer, followed by further centrifugation at 1350 rpm for 10 minutes. After discarding a supernatant again, the remaining product was suspended in 50 mL of the washing buffer, followed by measuring the number of cells to determine cell viability.

The supernatant was discarded by centrifugation at 1200 rpm for 10 minutes, and then, suspended in FBS to have a concentration of 4×10⁷ cells/mL. The product was mixed with the same volume of 20% (vol/vol) DMSO to prepare a final suspension in 10% DMSO. The suspension was seeded by 1.5 mL in a 2 mL cryogenical vial and frozen using a controlled-rate freezer (CRF), followed by storage in a liquid nitrogen tank.

### EXAMPLE 1-2

### DC:T Cell Assay

1. Procedures for Preparation of Dendritic Cells

1.1. PBMC Thawing

After quickly unfreezing cryopreserved PBC in a water bath at 37° C., the solution was moved to a 50 mL conical tube. While whirling the tube, a thawing medium (RPMI supplemented with 10% (vol/vol) FBS) was added dropwise and mixed well (15 mL/vial). After centrifugation at 1200 rpm for 10 minutes, a supernatant was discarded and the product was suspended in 30 mL of MACS buffer (PBS supplemented with 0.5% (vol/vol) FBS and 2 mM EDTA). After measuring the number of cells to determine cell viability, a supernatant was discarded by centrifugation at 1200 rpm for 10 minutes.

1.2. Monocyte Isolation

CD14 microbeads (Miltenyi Biotech) were added to the thawed PBMC and stained in ice for 15 minutes. After adding 30 mL of MACS buffer to the above product and centrifuging the same at 1350 rpm for 8 minutes, a supernatant was discarded. After suspending the product in 500 μL MACS buffer, CD14+ cells were isolated through LS column (Miltenyi Biotech). The resultant product was subject to measurement of the number of cells to determine cell viability, and then, prepared by centrifugation at 1350 rpm for 8 minutes.

1.3. DC Differentiation

The isolated CD14+ cells were suspended in RPMI medium (supplemented with 10% (vol/vol) FBS) to have a concentration of 6×10⁵ cells/mL. This suspension was put into 24-well plate by 1 mL per well. Thereafter, 1000 U/mL GM-CSF (R&D systems) and 1000 U/mL IL-4 (R&D systems) were added thereto, followed by culturing the same in a CO₂ incubator at 37° C. for 5 days.

Day 5 of the cultivation, 700 μL of supernatant was removed from each 24-well by a pipette, instead, a medium containing cytokine was added by 1 mL. The concentration of each cytokine is as follows: 1000 U/mL GM-CSF (R&D systems); 1000 U/mL IL-4 (R&D systems); 10 ng/mL TNF-α (R&D systems); 10 ng/mL IL-1β (R&D systems); 10 ng/mL IL-6 (R&D systems); 1 μg/mL PGE2 (Prostaglandin E2, Sigma). A protein agent with immunogenicity to be evaluated was put into each 24-well to an appropriate concentration of 0.01 to 1.0 μM (e.g. 0.3 μM). This agent was cultured in the CO₂ incubator at 37° C. for 2 days.

1.4. DC Assay

DC cultured in each 24-well for 7 days was moved to a 5 mL tube by a pipette. A portion of the recovered DC was evaluated through flow cytometry to determine whether the differentiation is adequately performed. It could be identified that the expression of CD14 disappeared during differentiation of monocyte into DC, whereas the expression of CD209 was increased. Further, differentiation into mature DC was assessed by determining whether the expression of HLA-DR, CD80, CD83, CD86, etc. was increased.

The recovered DC was subjected to measurement of the number of cells to determine cell viability, and 3 mL of AIM-V medium was added thereto to prepare a suspension at 5×10⁵ cells/mL. γ-irradiation was conducted at 2000 cGy to prepare DC for DC:T cell assay.

2. Procedures of T Cell Preparation

2.1. PBMC Thawing

After quickly unfreezing the cryopreserved PBC in a water bath at 37° C., the solution was moved to a 50 mL conical tube. While whirling the tube, a thawing medium was added dropwise and mixed well (15 mL/vial). After centrifugation at 1200 rpm for 10 minutes, a supernatant was discarded and the remaining product was suspended in 30 mL of MACS buffer. After measuring the number of cells to determine cell viability, a supernatant was discarded again by centrifugation at 1200 rpm for 10 minutes.

2.2. Depletion of CD8+ T Cells

CD8 microbeads (Miltenyi Biotech) were added to the thawed PBMC and stained in ice for 15 minutes. After adding 30 mL of MACS buffer to the above product and centrifuging the same at 1350 rpm for 8 minutes, a supernatant was discarded. After suspending the product in 500 μL MACS buffer, CD8+ cells were isolated through LS column (Miltenyi Biotech). The obtained CD8-cells were subject to measurement of the number of cells to determine cell viability, and then, prepared by centrifugation at 1350 rpm for 8 minutes.

2.3. VPD450 Staining of CD8-Cells

After discarding the supernatant, CD8-cells were suspended in 1× D-PBS and prepared so as to have a concentration of 2×10⁷ cells/mL. VPD450 solution (1 μM VPD450 in 1×DPBS) was added in the same volume as that of 1×D-PBS and mixed well to prepare a suspension, followed by culturing the same in a CO₂ incubator at 37° C. for 15 minutes. 10 mL of the thawing medium was added to the suspension, followed by centrifugation at 1200 rpm for 10 minutes. After discarding a supernatant, the remaining product was suspended in 12 mL of AIM-V medium and then subjected to measurement of the number of cells to determine cell viability. After centrifuging at 1200 rpm for 10 minutes and discarding a supernatant, AIM-V medium was added so as to have a concentration of 5×10⁶ cells/mL.

3. T Cell Proliferation Assay

3.1. DC: T Cell Plating

The DCs and T cells prepared in sections 1.4 and 2.3, respectively, were plated in 96-well plate by 100 μL for each stimulant condition in triplicate. The product was cultured in a CO₂ incubator at 37° C. for 7 days.

3.2. Flow Cytometric Analysis

The cells cultured for 7 days were stained with a fluorescent-labelled antibody, as shown in Table 1 below, thus to selectively measure proliferation of CD4+ T cells.

TABLE 1
Antibody mixture

| Fluorochrome | FITC | PE | PerCP | APC |
|---|---|---|---|---|
| Antibody | CD3 | CD8 | 7-AAD | CD4 |

More particularly, the cultured cells were further cultured along with the antibody mixture in ice for 30 minutes. After centrifugation at 2000 rpm for 3 minutes, a supernatant was discarded. 200 μL of FACS buffer (FACS sheath solution (BD) supplemented with 1% (vol/vol) FBS) was added, followed by washing the same. This process was repeated twice. The resultant product was suspended in 200 μL of BD CytoFix™ and then moved to 1.1 mL tubes (Axygen). Proliferation of CD4+ T cells were assayed by flow cytometry (BD).

### EXAMPLE 2

### Verification of Immunogenicity Determining Method

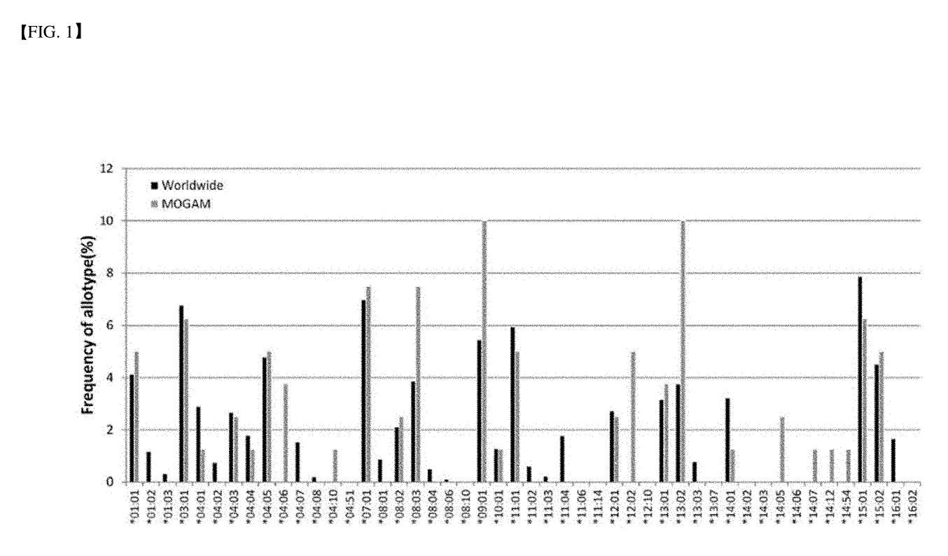

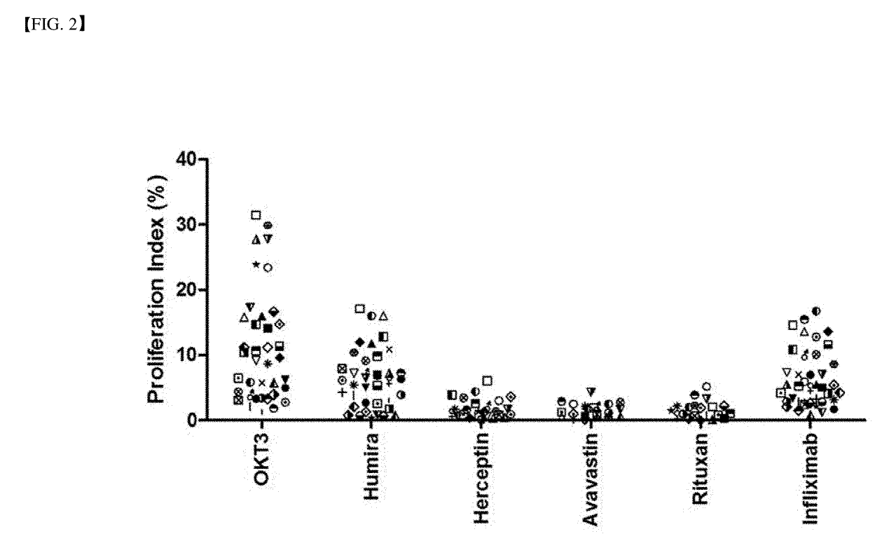

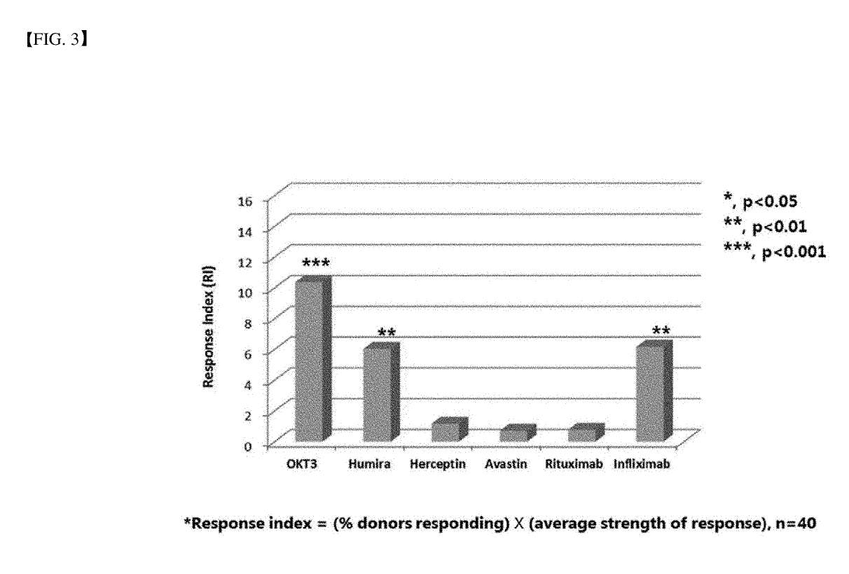

In order to verify the method for determination of immunogenicity according to the present invention, this method was compared to a world population distribution with reference to HLA-DRB1 allotype. Following this, PBMC in 40 or more different donors corresponding to about 80% coverage thereof was used as a target (FIG. 1). Six (6) types of protein agents (Table 2) with ADA generation extent identified in a clinical phase were assayed by the method for determination of immunogenicity according to the present invention. FDA-approved antibody therapeutic agents with immunogenicity reported in the art were summarized in Table 2 below. Further, assay results of immunogenicity according to the present invention were briefly illustrated in FIGS. 2 and 3.

TABLE 2

| Antibody | | | | | Reported | |
|---|---|---|---|---|---|---|
| name | Company | Type | Target | Indication(s) | immunogenicity* | |
| Muromanab | Ortho Biotech | Murine | CD3 | Allograft rejection | 47% | (50) |
| (OKT3) | | | | | | |
| Adalimumab | Abbott | Human | TNFα | RA/Crohn/PsA/JIA/ | 2.6%-26% | |
| (Humira) | | | | Ankylosing | | |
| | | | | spondylitis/plaque | | |
| | | | | psoriasis | | |
| Trastuzumab | Genentech | Humanized | Her2/neu | Breast cancer | <1% | |
| (Herceptin) | (Roche) | | | | | |
| Bevacizumab | Genentech | Humanized | VEGF | Colorectal, breast, | 0% | (−500) |
| (Avastin) | (Roche) | | | renal and NSCL | | |
| | | | | cancer | | |
| Rituximab | Genentech | Chimeric | CD20 | Non-Hodgkin | 11% | (2568) |
| (Rituxan) | (Roche)/Biogen | | | lymphoma | | |
| | Idec | | | | | |
| Infliximab | Centocor | Chimeric | TNFα | RA/Crohn | 25% | (99) |
| (Remcicade) | (Johnson & | | | | | |
| | Johnson) | | | | | |

*Frequency (size of patient group)

FIG. 2 illustrates a proliferation index (PI) drawn by the determination method of the present invention. The PI refers to a value obtained by removing the background proliferative response from proliferative response of each protein agent with respect to PBMC of 40 donors participated in the assay.

FIG. 3 illustrates a response index (RI) drawn by the determination method of the present invention. The RI refers to a value obtained by multiplying % of donors positively responding to each protein agent among total donors and PI for each protein agent. The larger the response index (RI) means the higher immunogenicity of the protein agent. Further, as a result of performing ANOVA analysis with respect to PI values greater than 2× SEM value of the background proliferative response for each donor PBMC, the protein agent with a high RI value has been identified to exhibit statistical significance, compared to a negative control group. On the other hand, the protein agent with a low RI value did not show statistical significance, compared to the negative control group.

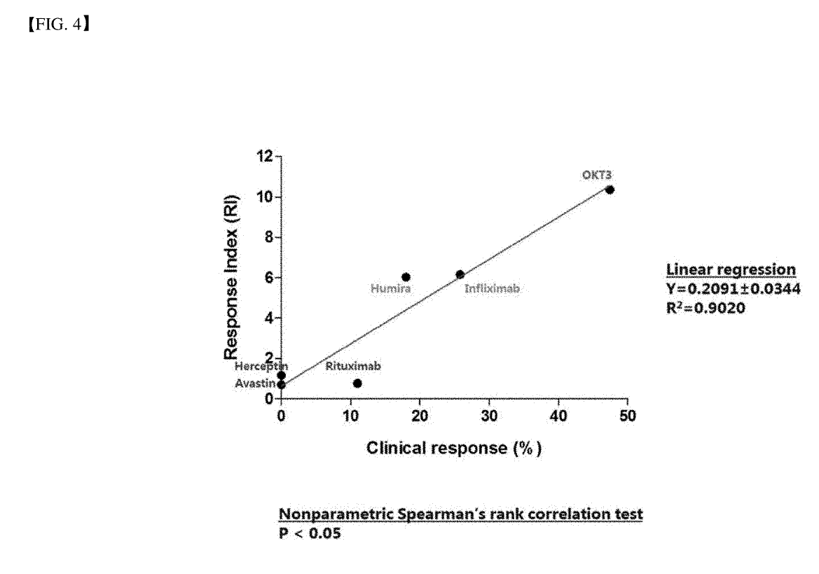

FIG. 4 illustrates a correlation of the response index (Y-axis) deduced by the determination method of the present invention and an ADA generation index (X-axis) clinically reported in the art, which exhibits statistical significance such as p value<0.05, R²>0.9. Therefore, it can be seen that the method for determination of immunogenicity according to the present invention has a high correlation with the ADA generation extent clinically reported in the art. This means that, when the immunogenicity determination method of the present invention which measures the immunogenicity of a specific protein agent in vitro (ex vivo) is further applied to a clinical practice, it is possible to predict how much ADA is produced in the body (that is, the level of immunogenicity to be achieved) with a high accuracy.

1. A method for determining immunogenicity of a protein agent, comprising:
constructing a library of peripheral blood mononuclear cells having various HLA-DRB1 genotypes;
culturing peripheral blood mononuclear cell CD14+ monocyte-derived immature dendritic cells for each genotype in a medium containing the protein to be measured, GM-CSF, IL-4, TNF-α, IL-1β, IL-6 and PGF₂ to prepare mature dendritic cells;
removing CD8+ T cells from the peripheral blood mononuclear cells for each genotype to prepare CD8+ T cell-free peripheral blood mononuclear cells;
co-culturing the mature dendritic cells and the CD8+ T cell-free peripheral blood mononuclear cells at a cell count ratio of 1:5 to 1:20; and
quantifying the number of CD4+ T cells proliferated by the co-cultivation per genotype.

2. The method according to claim 1, wherein the peripheral blood mononuclear cell library includes 70% or more of human HLA-DRB1 genetic polymorphism.

3. The method according to claim 1, wherein the peripheral blood mononuclear cells are extracted from the blood collected by Leukoreduction system chambers (LRSCs).

4. The method according to claim 1, wherein the immature dendritic cells are prepared by culturing the CD14 + mononuclear cells in a medium containing GM-CSF and IL-4.

5. The method according to claim 1, wherein the CD8+ T cell-free peripheral blood mononuclear cells provided for the co-cultivation are stained with a fluorescent dye, and the quantification includes quantifying the number of CD4+ T cells having a fluorescent signal intensity decreased after the co-cultivation.

6. The method according to claim 1, wherein the medium in the co-cultivation is a serum-free medium further containing L-glutamine, human serum albumin, streptomycin sulfate and gentamicin sulfate.

7. The method according to claim 6, wherein the streptomycin sulfate is contained in an amount of 50 μg/ml, and the gentamicin sulfate is contained in an amount of 10 μg/ml.

8. The method according to claim 1, wherein the medium in the step of preparing the mature dendritic cells further includes Ca(NO₃)₂.4H₂O, KCl, MgSO₄ (anhydrous), NaCl, Na₂HPO₄ (anhydrous), D-glucose, Glutathione (reduced), Phenol red, L-arginine, L-asparagine (free base), L-aspartic acid, L-cystine⋅2HCl, L-glutamic acid, L-glutamine, glycine, L-histidine (free base), L-hydroxyproline, L-isoleucine, L-leucine, L-lysine⋅HCl, L-methionine, L-phenylalanine, L-proline, L-serine, L-threonine, L-tryptophan, L-tyrosine⋅2Na2H₂O, L-valine, biotin, D-Ca⋅pantothenate, choline chloride, folic acid, i-inositol, para-aminobenzoic acid, niacinamide, pyridoxine⋅HCl, riboflavin, thiamine⋅HCl and vitamine B12, and further includes 10% (vol/vol) FBS.

9. The method according to claim 4, wherein the medium used in the step of preparing the immature dendritic cells further include Ca(NO₃)₂.4H₂O, KCl, MgSO₄ (anhydrous), NaCl, Na₂HPO₄ (anhydrous), D-glucose, Glutathione (reduced), Phenol red, L-arginine, L-asparagine (free base), L-aspartic acid, L-cystine⋅2HCl, L-glutamic acid, L-glutamine, glycine, L-histidine (free base), L-hydroxyproline, L-isoleucine, L-leucine, L-lysine⋅HCl, L-methionine, L-phenylalanine, L-proline, L-serine, L-threonine, L-tryptophan, L-tyrosine⋅2Na2H₂O, L-valine, biotin, D-Ca⋅pantothenate, choline chloride, folic acid, i-inositol, para-aminobenzoic acid, niacinamide, pyridoxine⋅HCl, riboflavin, thiamine⋅HCl and vitamine B12, and further includes 10% (vol/vol) FBS.

10. A kit for measuring immunogenicity of a protein agent, comprising a library of peripheral blood mononuclear cells including 70% or more of human HLA-DRB1 genetic polymorphisms.
